(19) United States
(12) Patent Application Publication
(10) Pub. No.: US 2004/0191243 A1
(43) Pub. Date: Sep. 30, 2004

(54) System and method for stabilizing antibodies with histidine

(76) Inventors: Bei Chen, Fremont, CA (US); Gerardo Zapata, San Mateo, CA (US); Michael G. Mulkerrin, Hillsborough, CA (US); Steven M. Chamow, San Mateo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/734,606

(22) Filed: Dec. 11, 2003

Related U.S. Application Data
(60) Provisional application No. 60/433,546, filed on Dec. 13, 2002.

(51) Int. Cl. A61K 39/395

(52) U.S. Cl. 424/130.1; 514/400

(57) ABSTRACT

Stabilized antibody formulations containing histidine are described. In addition, methods of stabilizing liquid formulations of antibodies using histidine are also described. Kits using histidine to stabilize histidine.

### RELATED APPLICATIONS

This application claims priority under 35 U.S.C §119(e) to U.S. Provisional Application No. 60/433,546, filed Dec. 13, 2002, which is hereby expressly incorporated by reference in its entirety.

### BACKGROUND OF THE INVENTION

1. Field of the Invention

The present invention relates to stabilized antibody formulations and methods of stabilizing antibodies. In certain aspects, the invention relates to the use of histidine as a cryoprotectant and lyoprotectant. The invention also relates to kits for stabilizing antibodies in liquid and solid formulations.

2. Description of the Related Art

With the tremendous advancement of biotechnology and genomic sciences, antibodies are becoming very important as therapeutics. In order to successfully market an antibody, the product should have an adequate shelf life. Developing stable antibody dosage forms presents significant challenges because antibodies are susceptible to being degraded by a wide variety of pathways.

The two general degradation pathways that can effect an antibody are physical and chemical degradations. Physical degradations are changes in higher order protein structures (secondary, tertiary and quaternary) and do not involve covalent modification of the protein. Examples of physical degradations include aggregation, adsorption, denaturation and precipitation. In contrast, chemical degradations involve modification of the primary structure of proteins via bond formation or cleavage, thereby yielding a new chemical entity. Examples of chemical degradations include deamidation, isomerization, oxidation and hydrolysis. While technically distinct, physical and chemical degradations are often interrelated. For example, a partially unfolded protein (physically degraded) can result in an increase in oxidation (chemical degradation).

Among the many ways to formulate antibody drugs, liquid and lyophilized (powdered) dosage forms are some of the more common formulations used today. Some of the advantages liquid dosages have over lyophilized formulations are that they are less expensive and generally easier to administrate. Lyophilized formulations are usually preferred over liquid formulations when storing antibodies at a high concentration. Furthermore, since lyophilized formulations are dry, they are generally more stable and have slower degradation rates than liquid formulations. Nevertheless, lyophilization involves freezing and drying steps, both of which can induce stress on an antibody. For example, antibodies are susceptible to structural unfolding during the freezing process. In addition, the drying process can alter the secondary structure of an antibody molecule. Damage to dried antibodies can be manifested after rehydration as a loss of protein solubility, aggregation, loss of activity in appropriate biosassays, and loss of antibody purity. Accordingly, the use of cryoprotectants and lyoprotectants in a lyophilized formulation can be highly beneficial in preventing degradation of antibodies.

Traditionally, sugars have been extensively studied for use as cryoprotectants and lyoprotectants in stabilizing proteins against denaturation to prevent aggregation during freezing and lyophilization. For example, it has been reported that hydrogen bonding between sugar and protein is responsible for inhibiting dehydration-induced protein unfolding (U.S. Pat. No. 5,358,708). Furthermore, it has been demonstrated that a specific molar ratio (360:1) of sugars (sucrose or trehalose) to protein is required for storage stability of a lyophilized monoclonal antibody. (U.S. Pat. No. 5,763,401).

While the traditional use of additives has improved the stability of dried proteins, many proteins which are subject to drying and subsequent storage contain unacceptable or undesirable amounts of inactive, aggregated protein in the rehydrated formula. While the prior art has disclosed stabilizing dried proteins upon rehydration by including reconstituting agents or osmolytes (U.S. Pat. No. 5,580,856), there has not been any disclosure that establishes histidine's usefulness in protecting the protein or antibody from the stresses of freezing and drying that accompany lyophilization.

Accordingly, there is a need in the art to establish a method of protecting antibodies from the stresses of lyophilization.

### SUMMARY OF THE INVENTION

Embodiments of the invention relate to solid formulations including at least one antibody, and histidine in a sufficient amount to stabilize said at least one antibody in said solid formulation. In certain embodiments the solid formulations can include at least one other excipient. For example, the solid formulation can include at least one other excipient selected from the group consisting of: mannitol, Polysorbate 20, Polysorbate 80, succinate, citrate, Tris, phosphate, trehalose, amino acids, polyols, PEG, BSA, sucrose, lactose, maltose, and sorbital. Further embodiments include solid formulations wherein the other excipient is arginine.

Other embodiments relate to solid formulations including a mammalian antibody. In further embodiments the antibody can be a human antibody. In other embodiments the antibody can be a human monoclonal IgG₂ antibody.

Any amount of histidine, sufficient to stabilize at least one antibody, can be used with the solid and liquid formulations described herein. In certain embodiments, the sufficient amount of histidine is between 6-40 mM. In other embodiments the sufficient amount of histidine is about 15 mM.

Further aspects of the invention include kits for preparing solid formulations of a stabilized antibody. Kits can include a first container, comprising at least one antibody in solution, and a second container comprising a sufficient amount of histidine in solution to stabilize said antibody when said antibody is dried into a solid formulation.

Any amount of histidine, sufficient to stabilize at least one antibody, can be used with the kits described herein. In certain embodiments, the sufficient amount of histidine is between 6-40 mM. In other embodiments the sufficient amount of histidine is about 15 mM.

Additional aspects relate to methods of preparing an antibody in a solid formulation. Methods can include mixing at least one antibody with a stabilizing amount of histidine to form a mixture; and treating said mixture to generate a solid formulation of said antibody. In certain embodiments, the mixture can include at least one other additional excipient. For example, the mixture can include at least one or more of the following excipients: mannitol, Polysorbate 20, Polysorbate 80, succinate, citrate, Tris, phosphate, trehalose, amino acids, polyols, PEG, BSA, sucrose, lactose, maltose, and sorbital. In other embodiments the excipient can be arginine.

Embodiments of the invention also include lyophilizing the mixture to generate a solid formulation, such as a lyophilized cake. Other embodiments relate to methods of reconstituting the lyophilized cake with a reconstituting agent. The reconstituting agent can include sterile water for injection, for example. Of course, other well-known reconstituting agents are within the scope of the invention

Other embodiments provide methods of lyophilizing a mixture of histidine and antibodies. These methods can include freezing the mixture at a rate of about 1° C. per minute until the mixture reaches a temperature of about −45° C.; and sufficiently drying the mixture. The drying step can include a primary and secondary drying step. In some embodiments, the lyophilization of the mixture occurs in less than 100 hours The lyophilization can also occur in less than 50 hours and even less than 45 hours.

Further embodiments of the invention relate to methods of stabilizing a mammalian antibody. In particular embodiments the antibody can include a human antibody or a human monoclonal IgG₂ antibody, for example.

Any stabilizing amount of histidine can be used with the methods described herein. In certain embodiments, the stabilizing amount of histidine is between 6-40 mM. In other embodiments the stabilizing amount of histidine is about 15 mM.

Embodiments of the invention also relate to liquid formulations including at least one antibody, and histidine in a sufficient amount to stabilize said at least one antibody in said liquid formulation. In certain embodiments, the liquid formulations can include at least one other excipient. For example, the liquid formulation can include at least one other excipient selected from the group consisting of: mannitol, Polysorbate 20, Polysorbate 80, succinate, citrate, Tris, phosphate, trehalose, amino acids, polyols, PEG, BSA, sucrose, lactose, maltose, and sorbital. Further embodiments include liquid formulations wherein the other excipient is arginine.

Other embodiments relate to liquid formulations including a mammalian antibody. In further embodiments, the antibody can be a human antibody. In other embodiments, the antibody can be a human monoclonal IgG₂ antibody.

### BRIEF DESCRIPTION OF THE DRAWINGS

FIG. 1 is a bar graph that shows the effect of increased concentrations of histidine and optimal freeze-drying cycles on reconstitution time of lyophilized formulations. The first formulation includes 50 mg/mL ABX-IL8, 15 mM histidine, 15 mM arginine, 25 mM sucrose, 10 mM mannitol, 0.025% polysorbate 20, pH 6.0. The second formulation includes 50 mg/mL ABX-IL8, 5 mM histidine, 17.5 mM glycine, 0.25% mannitol, 18.8 mM glutamic acid, and 0.025% polysorbate 20, pH 6.0. The second formulation was freeze-dried according to the freeze-drying cycle of Table 1B, and the first formulation was freeze-dried according to various shorter cycles. With respect to the first formulation, the bar on the left indicates that the reconstitution time was measured shortly after lyophilization, the middle bar indicates that the reconstitution time was measured 2 months after lyophilization at an incubation temperature between 2-8° C., and the bar on the right indicates that the reconstitution time was measured 26 months after lyophilization at an incubation temperature between 2-8° C. Similarly, for the second formulation, the bar on the left indicates that the reconstitution time was measured shortly after lyophilization, the middle bar indicates that the reconstitution time was measured 2 months after lyophilization at an incubation temperature between 2-8° C., and the bar on the right indicates that the reconstitution time was measured 26 months after lyophilization at an incubation temperature between 2-8° C.

FIG. 2 is a point graph that compares the percentage of aggregates between the two formulations described above, in FIG. 1. After a period of days the percentage of aggregates in both the first and second formulations was determined by SEC-HPLC. The solid triangles pointing upward represent the second formulation, while all other symbols represent the first formulation lyophilized with various freeze-drying cycles.

FIG. 3 is a bar graph that shows the effect of histidine concentrations on the reconstitution time of lyophilized ABX-IL8 cakes, which were freeze-dried from bulk solutions containing 50 mg/mL ABX-IL8 in 4 mM histidine (left column) or 6 mM histidine (right column).

FIG. 4 is a bar graph that shows the effect of histidine on the formation of soluble aggregates as determined by SEC-HPLC. ABX-IL8 was lyophilized from bulk solutions containing 50 mg/IL ABX-IL8 in 4 mM histidine (solid column) or 6 mM histidine (hollow column).

FIG. 5 is a gel that shows the typical effect of histidine on the formation of High Molecular Weight (HMW) bands determined by non-reducing SDS-PAGE. ABX-IL8 was lyophilized from bulk solutions containing 50 mg/mL ABX-IL8 in 4 mM histidine (lanes 1, 2, 7, 8) and 6 mM histidine (lanes 3, 4, 5, 6). Lane 9 is the molecule weight standard.

FIG. 6 is a set of scanning electron micrographs illustrating freeze-dried ABX-IL8 in the presence of 6 mM histidine (6A) or 4 mM histidine (6B) in the pre-lyophilization bulk material. Magnification=X 100.

FIG. 7 is a line graph that shows the second derivative spectra of ABX-IL8 in lyophilized Formulation 1 from Table 1A (solid line) and Formulation 3 from Table 1A (dashed line).

FIG. 8 is a bar graph that compares the effect of histidine and sucrose on the formation of soluble aggregates determined by SEC-HPLC. ABX-IL8 was lyophilized from bulk solutions containing 50 mg/mL ABX-IL8 in 10 mM (solid column) or 15 mM (hollow column) concentrations of histidine or sucrose.

FIG. 9 is a point graph that shows a comparison of the effect of histidine/arginine and sucrose on the solution stability of ABX-IL8. Hollow symbols represent ABX-IL8 in formulation A from Table 5. Solid symbols represent ABX-IL8 in formulation B from Table 5. Circle symbols represent samples at 2-8° C. Diamond symbols represent samples at 25° C. Square symbols represent samples at 40° C.

FIG. 10 is a point graph that shows the correlation between aggregation percentage and molar ratio of excipient to antibody. The six squares represent (from left to right) Formulations C, D, E, F, G, and H from Table 5. The circles represent formulations with sucrose instead of histidine.

FIG. 11 is a bar graph that illustrates histidine's effectiveness in preventing aggregation in liquid antibody formulations. With reference to Table 5, the solid bar represents Formulation D (15 mM histidine), the hollow bar represents Formulation J (15 mM succinate) and the striped bar represents Formulation I (15 mM citrate).

FIG. 12 is a point graph that shows the effect of histidine (solid circle) or histidine/arginine (hollow square) on the solution viscosity of ABX-IL8.

### DETAILED DESCRIPTION

### Overview

The present invention generally relates to histidine-containing solid and liquid formulations that are useful for stabilizing antibodies. The invention is also directed to methods of using histidine to prepare stabilized solid state and liquid antibody formulations. Furthermore, embodiments of the invention relate to kits that use histidine to stabilize antibodies.

### Antibodies

While the included Examples described herein are directed to a fully human monoclonal IgG₂ antibody, the present invention is not limited to any particular type of antibody. The term “antibody”, as used herein, is to be construed broadly. In general, the term “antibody” can include any of a large number of proteins of high molecular weight that act specifically against an antigen in an immune response. Antibodies can be a specific immunoglobulin from the classes IgA, IgD, IgE, IgG, IgM and subclasses thereof.

The term “antibody” also encompasses analogs thereof. In particular, complementarity determining regions (CDRs) are required, along with sufficient portions of the framework (Frs) to result in the appropriate three dimensional conformation. Typical immunospecific analogs of antibodies include F(abl″)₂, Fab′, and Fab regions. Modified forms of the variable regions to obtain, for example, single chain Fᵥ analogs with the appropriate immunospecificity are known. A review of such Fᵥ construction is found, for example, in Huston et al., Methods in Enzymology 203:46-63 (1991). The construction of antibody analogs with multiple immunospecificities is also possible by coupling the variable regions from one antibody to those of second antibody.

Embodiments of the invention are useful in stabilizing any type of antibody. In one aspect of the invention, the antibody can be supplied from any mammal. The following is a non-exclusive list of mammals that can be suitable providers of an antibody according to the present invention: rats, mice, dogs, cats, rabbits, pigs, goats, sheep, cattle, horses, and primates including monkeys, apes and humans. The antibodies produced can be obtained from the animal directly or from immortalized B-cells derived from the animal. Both monoclonal and polyclonal antibodies can be stabilized according to the methods described herein. Antibodies generated from non-animal systems can also be used (e.g., plant and yeast systems). In addition, formulations that include recombinant antibodies produced by well known methods are also within the scope of the invention.

In particular embodiments, antibodies can be generated by transgenic animals that have been genetically altered to produce exogenous antibodies. For example, a fully human monoclonal IgG₂ antibody can be generated using Abgenix's XenoMouse technology (Abgenix, Inc., Fremont, Calif.). One such antibody is ABX-IL8, which has kappa light chains and a molecular weight of approximately 150 kD with a pI range of about 7.3-8.5. The ABX-IL8 antibody is specific for interluekin-8 (IL8), a potent chemotactic cytokine with Kd of 2.1×10⁻¹⁰ M. The XenoMouse technology is described in detail in U.S. Pat. No. 6,150,584, entitled “Human Antibodies Derived From Immunized Xenomice” which is hereby incorporated by reference in its entirety. In general XenoMouse technology involves transgenic mouse strains possessing an immune system in which the mouse antibody-producing genes have been inactivated and functionally replaced by most of the human antibody-producing genes.

Any concentration of antibody can be stabilized according to the methods described herein. For example, the following concentrations of antibody can be stabilized either in liquid or solid histidine containing formulations: about 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL and more.

### Histidine and Other Excipients

Histidine is a unique amino acid. There are three ionization sites on the molecule, with pK₁′ of 1.78, pK₂′ of 5.97, and pK₃′ of 8.97. As mentioned earlier, the formulations, kits, and methods described are directed to using “sufficient amounts” of histidine to stabilize at least one antibody in a formulation.

The terms “sufficient amount” and “stabilizing amount” are interchangeable and refer to the amount of histidine added to a liquid formulation containing at least one antibody. For embodiments directed to solid formulations, kits for preparing solid formulations, and methods of preparing an antibody in a solid formulation, the terms “sufficient amount” and “stabilizing amount” refer to the amount of histidine that is added to a liquid formulation, prior to treating (e.g., lyophilizing) the liquid formulation to generate a solid formulation. Accordingly, the terms “sufficient amount” and “stabilizing amount” do not necessarily refer to the amount of histidine actually present in the solid formulation after treatment.

Solid state and liquid formulations that included sufficient amounts of histidine were found to effectively stabilize antibodies. The following is a non-exhaustive list of concentrations of histidine that can be used to stabilize antibodies: about 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM and more. Again, these amounts refer to the concentration of histidine present in a liquid formulation containing at least one antibody.

As described herein, stabilizing antibodies generally relates to retaining the antibody in its natural state or inhibiting antibody degradation. Accordingly, in certain embodiments stabilizing antibodies refers to inhibiting aggregate formation of antibodies, particularly during freezing and drying steps. It should be noted that antibody aggregation is dependent upon the antibody storage conditions, such as the length of storage and the storage temperature. Accordingly, skilled artisans will readily take these factors into account when assessing the stability profile of an antibody formulation. Depending on the storage conditions, in certain embodiments, stabilized formulations can include less than about 2% aggregation as determined by SEC-HPLC.

In other embodiments, stabilizing antibodies refers to inhibiting high molecular weight (HMW) bands. The presence of HMW bands in antibody formulations is also dependent upon the storage conditions. Accordingly, skilled artisans will readily take these factors into account when assessing the stability profile of an antibody formulation. Depending on the storage conditions, in certain embodiments, stabilized formulations can include less than about 3.2% HMW bands.

In some embodiments, the histidine containing formulations include one or more additional ekcipients. The term “excipient” is to be construed broadly and includes any additive that is suitable to be included in a stabilized antibody formulation. For example, histidine can be added with any of the following classes of excipients: buffers, cryoprotectants, lyoprotectants, bulking agents, surfactants and the like. Examples of suitable buffers include succinate, citrate, Tris, phosphate and the like. Examples of suitable cryoprotectants include sucrose, trehalose, polyols, polyethylene glycol (PEG), Bovine Serum Albumin (BSA), glutamic acid, other amino acids and the like. Suitable lyoprotectants can encompass sugars including sucrose, trehalose, lactose, and maltose and the like. Suitable bulking agents include mannitol, glycine, and sorbital and the like. Examples of possible surfactants include, polysorbate 20 polysorbate 80 and the like.

### Solid Formulations

In certain embodiments, the present invention includes antibodies stabilized in solid formulations. Solid formulations can include dried formulations, which encompasses formulations that have been subjected to spray-drying or air-drying. In other embodiments, dried formulations include lyophilized formulations, such as lyophilized cakes and the like.

Any lyophilization method known in the art is intended to be within the scope of the invention. In general, lyophilization includes at least one freezing process and at least one drying process. In other embodiments, lyophilization includes more than one freezing step and more than one drying step. For example, lyophilization can include about 1, 2, 3, 4, and 5 or more freezing steps, and about 1, 2, 3, 4, and 5 or more drying steps. In particular embodiments, the freezing step involves cooling the formulation from room temperature to −45° C. in about two hours. In a another embodiment, the freezing step involves cooling the formulation from room temperature to −45° C. at a rate of about 1° C./minute. It was found that antibody formulations which were dried using this freezing method had relatively quick reconstitution times.

In certain embodiments the drying process includes three steps. For example, a first drying step can take place at a ramping rate of 0.5° C. /min from −45° C. to −20° C. and then hold at −20° C. for 75 hours at a chamber pressure of 70 mTorr. A second drying step can take place at a ramping rate of 0.50° C. /min from −20° C. to 20° C. and hold at 20° C. for 44 hours at a chamber pressure of 50 mTorr. A third drying step can take place at 20° C. for 4 hours at a chamber pressure of 30 mTorr. This drying schedule is provided in Table 1B.

In some embodiments, another freeze-drying cycle is used. For example, a freezing step can take place at a ramping rate of 0.35° C./min to −45° C. where it is held for 5 hours at an ambient chamber pressure. A first drying step can take place at a ramping rate of 0.16° C./min from −45° C. to 20° C. where it is held at 20° C. for 25 hours at a chamber pressure of 200 mTorr. A second drying step can take place at a ramping rate of 0.5° C. /min from 20° C. to 30° C. where it is held for 10 hours at a chamber pressure of 50 mTorr.

Any reconstitution agent known in the art can be used to reconstitute the stabilized, solid state, antibody formulations described herein. Reconstituting agents can include osmolytes, various salts, water soluble synthetic and natural polymers, surfactants, sulfated polysaccharides, carrier proteins, buffers and the like. Suitable reconstituting agents are provided in U.S. Pat. No. 5,580,856 entitled “Formulation of a Reconstituted Protein, and Method and Kit for the Production Thereof”, which is incorporated by reference in its entirety.

### Liquid Formulations

In addition to being able to stabilize solid antibody formulations, histidine can also be used to stabilize liquid antibody formulations. Accordingly, histidine containing liquid formulations can be made using excipients readily known to those with skill in the art. For example liquid formulations can be prepared with buffers, surfactants, anti-oxidants, stabilizers and the like. In certain embodiments, liquid formulations are prepared using a TFF system with a Biomax 30 membrane (Millipore, Bedford, Mass.). Stabilized liquid formulations can be stored in any suitable containers. In certain embodiments, each liquid formulation of 0.8 mL can be dispensed into 3-mL Type 1 glass vials with 13-mm serum stoppers. Samples can be stored at any suitable temperature. Suitable temperatures can include about 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., and higher temperatures, for example.

The following Examples demonstrate the benefits of using histidine in solid and liquid antibody formulations. In particular these Examples show histidine can effectively stabilize both solid and liquid antibody formulations.

### EXAMPLE 1

### Preparation of Lyophilized Formulations for Study 1

As provided in Table 1A, ABX-IL8 antibodies were purified and buffer-exchanged into 8 different formulations (2⁷⁻⁴ fractional factorial design) using PD10 columns (Amersham Pharmacia Biotech, Uppsala, Sweden). Each 0.8-mL formulation was dispensed into 3-mL Type 1 glass vials with 1 3-mm lyophilization stoppers.

TABLE 1A
Eight Formulations Based on the 2⁷⁻⁴ Fractional Factorial Design Matrix for
Study 1. 50 mg/mL of antibody was used in each Formulation.

| | Factors | | | | | | |
|---|---|---|---|---|---|---|---|
| | Freeze | | | | | | |
| | Drying | | Glycine^{a} | Histidine | Mannitol | Glutamic | Polysorbate |
| Formulation | cycle | pH | (mM) | (mM) | (%) | Acid (mM) | 20 (%) |
| 1 | II | 6.3 | 15 | 4 | 0.175 | 16.25 | 0.02 |
| 2 | II | 6.3 | 20 | 4 | 0.325 | 21.25 | 0.03 |
| 3 | II | 5.7 | 15 | 6 | 0.175 | 21.25 | 0.03 |
| 4 | II | 5.7 | 20 | 6 | 0.325 | 16.25 | 0.02 |
| 5 | I | 6.3 | 15 | 6 | 0.325 | 16.25 | 0.03 |
| 6 | I | 6.3 | 20 | 6 | 0.175 | 21.25 | 0.02 |
| 7 | I | 5.7 | 15 | 4 | 0.325 | 21.25 | 0.02 |
| 8 | I | 5.7 | 20 | 4 | 0.175 | 16.25 | 0.03 |

^{a}Excipient concentrations were the concentrations of bulk drug solutions.

After preparation, the eight formulations shown in Table 1A were lyophilized as described in Table 1B. The formulations were frozen according to either Cycle I or Cycle II as indicated in Table 1B.

TABLE 1B
Lyophilization Cycles

| | | | Duration | Chamber Pressure |
|---|---|---|---|---|
| Steps | Ramping Rate | Temperature | (hours) | (mTorr) |
| Freezing* | | −45° C. | 2 | |
| 1^{st} drying | 0.5° C./min | −20° C. | 70 | 70 |
| 2^{nd} drying | 0.5° C./min | 20° C. | 44 | 50 |
| 3^{rd} drying | NA | 20° C. | 4 | 30 |

*Cycle I: precool the shelf to −45° C.,
Cycle II: freeze at 1° C./min to −45° C.

Lyophilization was carried out in a DuraDry MP freeze-dryer (FTS Systems, Stone Ridge, N.Y.). All lyophilized cakes in the study had a residual water content of approximately 1%. This excluded the possibility that the recorded stability profiles were atributable to differences in the water content of the lyophilized cakes. The lyophilized vials were stored at different temperatures for different intervals of time.

Each formulation was then reconstituted with 0.2 mL water for injection (WFI) prior to subsequent assays. These assays, which are explained in detail in further Examples include: 1) UV-Vis Spectophotometry 2) Measuring reconstitution time, 3) Size Exclusion Chromatography-High Performance Liquid Chromatography (SEC-HPLC), 4) Sodium Dodecyl Sulfate-Polyacrylamide Gel Electrophoresis (SDS-PAGE), 5) Scanning Electron Microscopy (SEM), and 6) Fourier-Transform Infrared Spectroscopy (FTIR).

### EXAMPLE 2

### Effect of Freeze-drying Cycles

A first formulation including 50 mg/mL ABX-IL8, 15 mM histidine, 15 mM arginine, 25 mM sucrose, 10 mM mannitol, 0.025% polysorbate 20 was lyophilized. A second formulation including 50 mg/mL ABX-IL8, 5 mM histidine, 17.5 mM glycine, 0.25% mannitol, 18.8 mM glutamic acid, and 0.025% polysorbate 20 was also lyophilized.

The first formulation was freeze-dried using the following schedule. First a freezing was conducted at a ramping rate of 0.35° C. /min until the shelf temperature reached −45° C. where it was held for 5 hours at an ambient Chamber pressure. A first drying was carried out at a ramping rate of 0.16° C. /min from −45° C. to 20° C. and held for 25 hours at a chamber pressure of 200 mTorr. Finally a second drying at a ramping rate of 0.5° C. /min from 20° C. to 30° C. and was held for 10 hours at a chamber pressure of 50 mTorr. In contrast, the second formulation was freeze-dried according to the freeze-drying cycle of Table 1B. Accordingly the first formulation was freeze-dried in approximately 45 hours while the second formulation was freeze-dried in about 125 hours. Both the first and the second formulation were reconstituted with W.F.I.

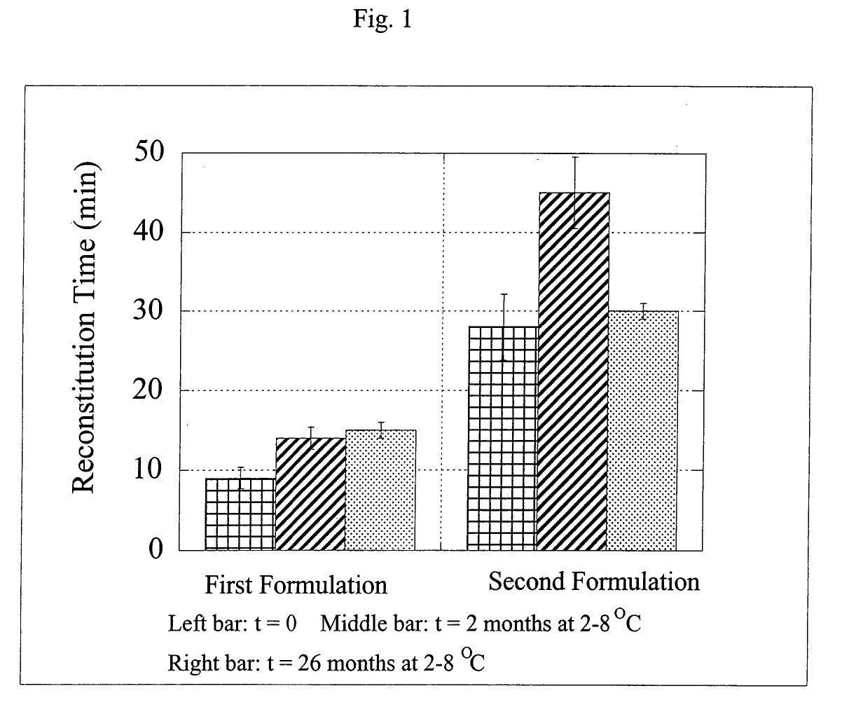

Reconstitution time was measured twice for both the first and second formulations. The first measurement was shortly after lyophilization and then 2 months afterwards at 2-8° C. FIG. 1 is a bar graph that shows the effect of increased concentrations of histidine and optimal freeze-drying cycles on reconstitution time of lyophilized formulations. Referring to FIG. 1, the bar on the left indicates that the reconstitution time was measured shortly after lyophilization, the middle bar indicates that the reconstitution time was measured 2 months after lyophilization at an incubation temperature between 2-8° C., and the bar on the right indicates that the reconstitution time was measured 26 months after lyophilization at an incubation temperature between 2-8° C. As FIG. 1 illustrates, the first formulation (15 mM histidine and a shortened freeze-drying cycle) reconstituted more rapidly than the second formulation (5 mM histidine and a longer freeze-drying cycle).

In addition to being prepared faster, the first formulation lyophilized cakes did not have any powder film on the wall of the vials, did not collapse, and were only slightly shrunken. In contrast, the second formulation lyophilized cakes had powder on their walls, some were collapsed, and they were more shrunken than the first formulation cakes. The first formulation cakes also had a lower residual moisture (about 1%) and lower monomer loss constant [0.1-0.3 (10⁻³ day⁻¹)] than the second lab formulations (about 3%) and [0.5 (10⁻¹ day⁻¹)].

### EXAMPLE 3

### Aggregation of Formulations

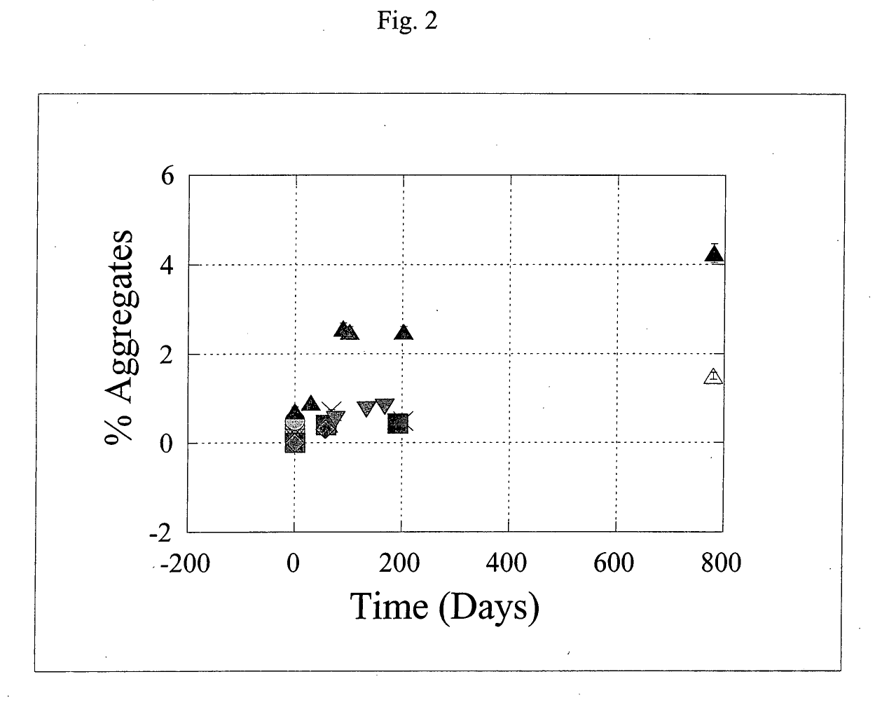

First and second formulations having the same excipients as described above, in Example 2 were prepared. The second formulation was freeze-dried according to the freeze-drying cycle of Table 1B which took approximately 125 hours. The first formulations were freeze-dried according to various shorter cycles. After storage at 2-8° C., the percentage of aggregates in both the first and second formulations was determined by SEC-HPLC. The results are provided in FIG. 2.

FIG. 2 is a point graph that compares the percentage of aggregates between first and second formulations (the same first and second formulations that are described in Example 15). The second formulation was freeze-dried according to the freeze-drying cycle of Table 1B. The first formulation was freeze-dried according to various shorter cycles. After a period of days the percentage of aggregates in both the first and second formulations were determined by SEC-HPLC (repeating of the previous paragraph, consider to delete). The triangles pointing upward represent the second formulation, while all other symbols represent the first formulation at various shorter freeze-drying cycles. The results show that the first formulation had lower levels of aggregates than the second formulation. Accordingly, the first formulation, with a higher concentration of histidine and a shorter freeze-drying period, had fewer aggregates than the second formulation, which had a lower concentration of histidine and a longer freeze-drying cycle.

### EXAMPLE 4

### Youden 2⁷⁻⁴ Fractional Factorial Design

A modified fractional factorial (2⁷⁻⁴) design, as described by Youden, was used to test the effects of seven different factors in the eight formulations described in Example 1. (W. J. Youden “Statistical techniques for collaborative tests,” Association of Official Analytical Chemists (AOAC), Arlington, Va.) Fractional factorial designs for screening purposes are useful in that they allow researchers to test many variables (factors) in a small number of experiments, identify critical formulation parameters effectively, rank the importance of each parameter on different responses, and to gain direction for further experiments.

The seven factors that were tested herein included: freeze-drying cycle, pH, glycine, histidine, mannitol, glutamic acid, and polysorbate 20. The Youden technique states that the effect of a factor on a response can be determined by taking the average of the responses at the higher level (+) minus the average of the response at the low (−) level. Effect=Δ (high-low)=(Σ responses on high setting/4)−(Σ responses on low setting/4).

Table 2A lists the seven factors chosen for the study, the levels at which they were tested, and which levels were indicated as high (+) or low (−) levels.

TABLE 2A
Experimental Factors and Levels

| | | Test Level I | Test Levels II |
|---|---|---|---|
| Factors | Target Value | (Low)(−) | (High)(+) |
| Freeze-drying cycle | | I | II |
| (X₁) | | | |
| pH | 6 | 5.7 | 6.3 |
| (X₂) | | | |
| Glycine (mM) | 20 | 15 | 20 |
| (X₃) | | | |
| Histidine (mM) | 5 | 4 | 6 |
| (X₄) | | | |
| Mannitol (%) | .25 | 0.175 | .325 |
| (X₅) | | | |
| Glutamic acid (mM) | 18.75 | 16.25 | 21.25 |
| (X₆) | | | |
| Polysorbate 20 | 0.025 | 0.02 | 0.03 |
| (X₇) | | | |

Table 2B indicates whether a particular formulation contained a specific factor at a high (+) or low (−) level.

TABLE 2B
Youden 2⁷⁻⁴ Fractional Factorial Design Matrix

Factors

| Formulation | X₁ | X₂ | X₃ | X₄ | X₅ | X₆ | X₇ |
|---|---|---|---|---|---|---|---|
| 1 | + | + | − | − | − | − | − |
| 2 | + | + | + | − | + | + | + |
| 3 | + | − | − | + | − | + | + |
| 4 | + | − | + | + | + | − | − |
| 5 | − | + | − | + | + | − | + |
| 6 | − | + | + | + | − | + | − |
| 7 | − | − | − | − | + | + | − |
| 8 | − | − | + | − | − | − | + |

The three assays included reconstitution time, percentage of High Molecular Weight (HMW) bands as determined by Sodium Dodecyl Sulfate-Polyacrylamide Gel Electrophoresis (SDS-PAGE), and percentage of Aggregates as determined by Size Exclusion Chromatography—High Performance Liquid Chromatography (SEC-HPLC).

As discussed above, aggregation of antibodies in lyophilized formulations can reduce the effectiveness of the antibodies when administered to a patient. Accordingly, it is important to minimize aggregation when reconstituting lyophilized antibodies. Both the SDS-PAGE and SEC-HPLC assays were useful in detecting unwanted antibody aggregation. Table 2C lists the results of these three assays.

TABLE 2C
Experimental Results of Example 1

| Factor | Formulation^{a} | | | | | | | |
|---|---|---|---|---|---|---|---|---|
| Set up | 1 | 2 | 3 | 4 | 5 | 6 | 7 | 8 |
| Freeze-drying | II | II | II | II | I | I | I | I |
| cycle | | | | | | | | |
| pH | 6.3 | 6.3 | 5.7 | 5.7 | 6.3 | 6.3 | 5.7 | 5.7 |
| Glycine(mM) | 15 | 17.5 | 15 | 17.5 | 15 | 17.4 | 15 | 17.5 |
| Histidine (mM) | 4 | 4 | 6 | 6 | 6 | 6 | 4 | 4 |
| Mannitol (%) | 0.175 | 0.325 | 0.175 | 0.325 | 0.325 | 0.175 | 0.325 | 0.175 |
| Glutamic | 16.25 | 21.25 | 21.25 | 16.25 | 16.25 | 21.25 | 21.25 | 16.25 |
| Acid (mM) | | | | | | | | |
| Polysorbate 20 | 0.02 | 0.03 | 0.03 | 0.02 | 0.03 | 0.02 | 0.02 | 0.03 |
| (%) | | | | | | | | |
| Results | | | | | | | | |
| Rec. time | 23.0 | 18.4 | 16.4 | 17.5 | 17.6 | 25.4 | 32.6 | 33.0 |
| (min.) | | | | | | | | |
| % HMW band | 4.4 | 3.3 | 0 | 0 | 0 | 0 | 2.1 | 2.9 |
| By SDS-PAGE | | | | | | | | |
| % Aggregates | 4.3 | 3.0 | 1.8 | 1.7 | 1.5 | 1.5 | 3.8 | 4.8 |
| by SEC-HPLC | | | | | | | | |

^{a}Samples were stored at 37° C. for 1 month.

In order to compare the effects of each factor on each response, factors were ranked in the order of relative significance on each response in Table 2D. For instance, for the factor histidine concentration, the effect on the response HMW band can be determined as follows.

Effect=(Σ formulation 3, 4, 5, 6 /4)−(Σ formulation 1, 2, 7, 8/4)=(0+0+0+0)/4−(4.4+3.3+2.1+2.9)/4=−3.2

Table 2D demonstrates that among five excipients tested (histidine, glycine, mannitol, glutamic acid and polysorbate 20), histidine was the most critical excipient for stability of the antibodies in a dried form. Increasing histidine concentration in the antibody formulations inhibited the increase of high molecular weight (HMW) species and aggregation upon lyophilization and storage. Furthermore, increasing histidine levels also facilitated reconstitution of lyophilized cakes. Accordingly, the stability of ABX-IL8 was found to be highly dependent on the concentration of histidine.

Table 2D. Effect of each formulation parameter on each response; order of significance of factors on reconstitution time (top); order of significance of factors on HMW band formation (middle); order of significance of factors on soluble aggregate formation (bottom).

Response

Reconstitution Time Factor

| | |
|---|---|
| Freeze-drying cycle | −8.3 |
| Histidine | −7.3 |
| pH | −3.8 |
| Polysorbate 20 | −3.3 |
| Mannitol | −2.9 |
| Glycine | 1.2 |
| Glutamic acid | 0.4 |

HMW Band Formation

| | |
|---|---|
| Histidine | −3.2 |
| Freeze-drying cycle | 0.7 |
| pH | 0.7 |
| Mannitol | −0.5 |
| Glutamic Acid | 0.1 |
| Glycine | 0.1 |
| Polysorbate 20 | 0.1 |

Soluble Aggregate Formation

| | |
|---|---|
| Histidine | −2.4 |
| Mannitol | −0.6 |
| Glutamic acid | −0.6 |
| pH | −0.5 |
| Freeze-drying cycle | −0.2 |
| Glycine | −0.1 |
| Polysorbate 20 | −0.1 |

Among the seven formulation factors, the freeze-drying cycle had the most significant influence on reconstitution time, followed by histidine concentration. In terms of HMW band intensity and soluble aggregate formation, the most important factor was histidine concentration. The utility of Table 2D lies in the ability to identify critical formulation parameters and serve as a troubleshooting guide. For example, if there is a problem upon reconstitution with a HMW band, consultation of Table 2D reveals that the most influential factor is histidine concentration. This allows a skilled practitioner to quickly pinpoint the most likely source of a problem and then adjust that parameter accordingly.

### EXAMPLE 5

### Reconstitution time

The ability to reconstitute lyophilized formulations quickly is advantageous in that it allows for a more convenient administration of the antibody and improved dosage accuracy. In general, it is desirable to obtain a completely dissolved therapeutic antibody as fast as possible.

ABX-IL8 was formulated at 50 mg/mL in the 8 different formulations shown in Table 2C and thereafter lyophilized to produce dried cakes. The lyophilized ABX-IL8 cakes were incubated at 37° C. for 1 month. Lyophilized cakes were then reconstituted using WFI. The reconstituted vials were gently swirled to allow the cakes to dissolve. The time for the cakes to completely dissolve was recorded as reconstitution time.

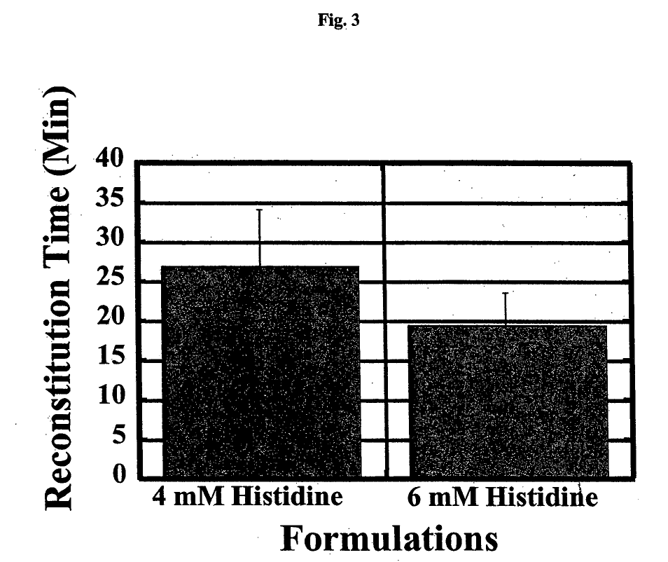

FIG. 3 is a bar graph that demonstrates that samples containing higher concentrations of histidine (6 mM) reconstituted much faster than those with lower level of histidine (4 mM). Referring to FIG. 3, the left column represents an average of Formulations 1, 2, 7, and 8 (50 mg/mL ABX-IL8 in 4 mM histidine) from Table 2C and the right column represents an average of Formulations 3, 4, 5, and 6 (50 mg/mL ABX-IL8 in 6 mM histidine) from Table 2C.

### EXAMPLE 6

### Size Exclusion Chromatography-High Performance Liquid Chromatography (SEC-HPLC)

The following experiment was used to determine the effect of histidine on the formation of soluble aggregates as measured by SEC-HPLC. ABX-IL8 was lyophilized from bulk solutions containing 50 mg/mL ABX-IL8 in 4 mM histidine and 6 mM histidine as provided in Table 1A. Three different incubation schedules were used to explore whether the ability of histidine to prevent aggregation was dependent upon incubation length or temperature. The three incubation schedules used were 1) 30 days at 37° C., 2) 150 days at 2-8° C. and 3) 180 days at 2-8° C. followed by 42 days at 25° C. The lyophilized cakes were reconstituted using WFI prior to testing.

Size exclusion chromatography was performed with a Water LC system coupled with a diode array detector. An TSK-Gel 3000 SW_{XL} column (0.78×30 cm; TosoHaas) was used with an elution buffer consisting of 500 mM sodium chloride, 50 mM borate, pH 8.0 with a flow rate of 0.5 mL/min. Mass load of the antibody was 50 μg and detection was at 215 nm.

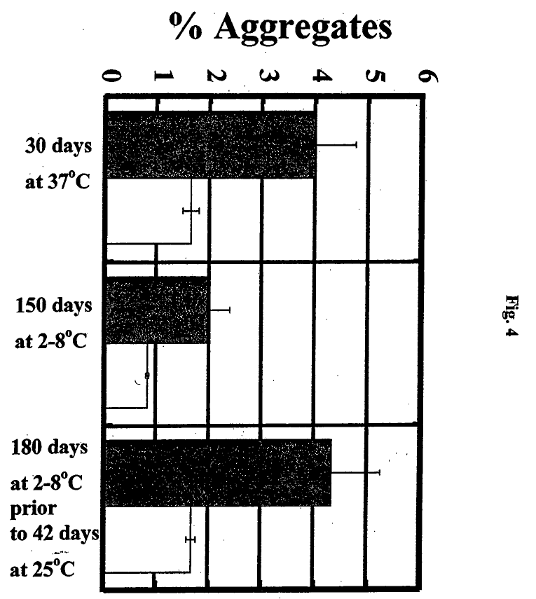

FIG. 4 is a bar graph that shows the effect of histidine on the formation of soluble aggregates as determined by SEC-HPLC assay. The results demonstrated that there was an inhibition of soluble aggregate formation for samples containing higher levels of histidine. As indicated by the hollow column, samples containing higher concentrations of histidine (6 mM) had significantly lower levels of aggregates than those samples containing lower concentrations of histidine (4 mM) as indicated by the solid column (P<0.01). Furthermore, these results are independent of the storage temperatures or length of incubation of the cakes. The solid bars represent an average of the 4 mM Formulations 1, 2, 7, 8 from Table 2C while the hollow bars represent an average of the 6 mM Formulations 3, 4, 5, 6 from Table 2C.

EXAMPLE 7

### Sodium Dodecyl Sulfate-Polyacrylamide Gel Electrophoresis (SDS-PAGE)

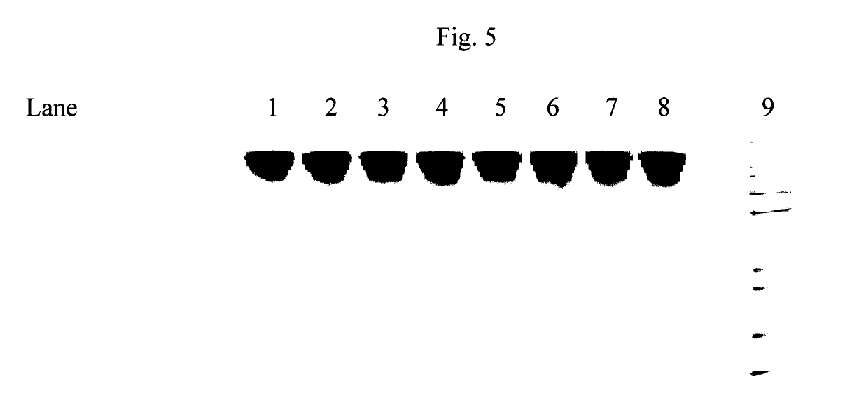

The following is the method that was used to examine antibody purity using SDS-PAGE. (See FIG. 5). ABX-IL8 was lyophilized from bulk solutions containing 50 mg/mL ABX-IL8 in 4 mM histidine (Formulations 1, 2, 7 and 8 from Table 2C) and 6 mM histidine (Formulations 3, 4, 5 and 6 from Table 2C). The lyophilized ABX-IL8 cakes were incubated at 2-8° C. for 6 months and subsequently at 25° C. for 42 days. Samples were reconstituted with Water for Injection (WFI).

SDS-PAGE was carried out on 10% Bis-Tris Novex ready gels (Invitrogen, Carlsbad, Calif.) using a Bio-Rad mini Protean II electrophoresis system. Samples were diluted with 2× glycine/SDS solution, either with or without DTT to a protein concentration of 0.5 mg/mL. Formulations were loaded into lanes (10 μg per lane) corresponding to their Formulation number (i.e. Formulation 1 was loaded into lane 1, Formulation 2 was loaded into lane 2, etc). Accordingly, lanes 1, 2, 7, and 8 contained corresponding 4 mM histidine formulations from Table 2C and lanes 3, 4, 5, and 6 contained corresponding 6 mM formulations from Table 2C. Lane 9 contained the molecule weight standard. Samples (10 μg per lane) were subjected to electrophoresis at 100 mA/gel current for approximately 45 minutes. Protein bands were visualized using Coomaassie blue followed by destaining until the backgrounds were clear. The intensity of the protein bands was determined by densitometry [AGFA Arcus II gel scanner (Scanalytics, Fairfax, Va.) with ONE-Dscan software] and calculated as a percentage of the total intensity of the sample.

Because HMW bands are indicative of unwanted aggregate antibodies, it is important to note that there were no high molecular weight (HMW) bands in the formulations with the 6 mM histidine concentrations after storage at 37° C. for 1 month, while there were an average of 3.2% HMW bands in the formulations with the 4 mM histidine concentrations. Accordingly, there was an inhibition of HMW formation in samples containing higher levels of histidine. FIG. 5 demonstrates a typical profile of a non-reducing SDS-PAGE gel. More specifically, FIG. 5 is a gel that shows the typical effect of histidine on the formation of High Molecular Weight (HMW) bands determined by non-reducing SDS-PAGE.

### EXAMPLE 8

### Scanning Electron Microscopy (SEM)

SEM was employed to examine the structure of the lyophilized antibody cakes. ABX-IL8 was freeze-dried in the presence of either 6 mM histidine (Formulation 5 from Table 2C) or 4 mM histidine (Formulation 8 from Table 2C). The lyophilized ABX-IL8 cakes were stored at 2-8° C. for 5 months.

Freeze-dried cakes were rapidly cut into pieces using a freshly made clean bamboo stick (Electron Microscopy Sciences, Pa.). The pieces were attached to a 12 mm OD aluminum SEM specimen-mounting stub by spreading a thin layer of the sample over a double-sided carbon conductive tab that was attached to the stub. This was performed rapidly (1-2 min) at room temperature to avoid adsorption of moisture by the samples. Then the sample stub was quickly transferred to a sputter coater and placed under vacuum. The samples were coated using a sputter coater (Biorad E5000M) with approximately 40 nm gold/palladium. Examination of samples was performed with a Hitachi S-A06 field emission SEM operating at 10 kv.

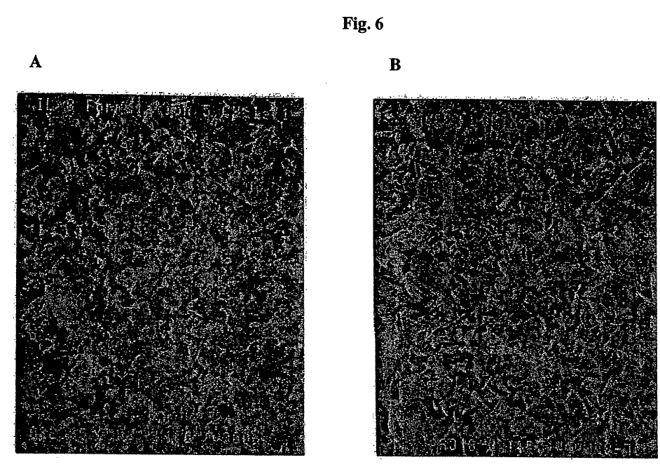

The results of the SEM are provided in FIG. 6. FIG. 6 is a set of scanning electron micrographs illustrating freeze-dried ABX-IL8 in the presence of 6 mM histidine (6A) or 4 mM histidine (6B) in the pre-lyophilization bulk material. Magnification=X 100. FIG. 6A shows that cakes lyophilized with higher concentrations of histidine (6 mM, Formulation 5 from Table 2C) exhibited a fine amorphous meshwork. In contrast, FIG. 6B shows that cakes with lower level of histidine (4 mM, Formulation 8 from Table 2C) exhibited a leafy structure. Furthermore, FIGS. 6A and 6B, show that cakes with higher concentrations of histidine (6 mM) had bigger pore sizes, which can allow more water to penetrate and result in a shorter reconstitution time.

EXAMPLE 9

### Fourier-Transform Infrared Spectroscopy (FTIR)

FTIR spectroscopy was used to probe the secondary structures of ABX-IL8 and the interactions between proteins and cosolvents in different formulation matrices.

Lyophilized Formulations 1 and 3 from Table 2C were measured as KBr pellets. A portion of 0.4 mg of lyophilized protein (ABX-IL8) was weighed out in a nitrogen purged dry box and each sample pressed into a pellet with 400 mg of KBr using a hydraulic press. The KBr pellet was scanned with a Bomem IR spectrophotometer. The data were collected in absorbance mode and background vapor was automatically subtracted. A total of 128 scans with a 4 cm⁻¹ resolution for each sample were averaged to obtain each spectrum. The resulting spectrum was smoothed with Bomem Grams 32 software (ABB Biomen, Inc., Quebec, Canada). Spectra were analyzed by second derivative to determine the number of spectral bands and their approximate locations. The spectral data were normalized with Prota software and imported into Igor Pro for analysis for secondary structure contents of the antibody.

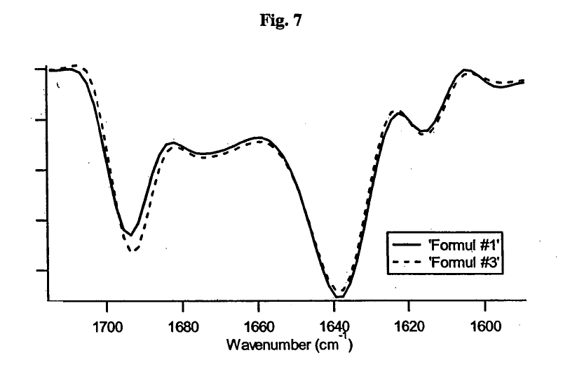

FIG. 7 is a line graph that shows the second derivative spectra of ABX-IL8 in lyophilized Formulation 1 from Table 1A (solid line) and Formulation 3 from Table 1A (dashed line). The lyophilized ABX-IL8 cakes were incubated for 5 months at 2-8° C. The results reveal that secondary structure profiles of Formulation 1 and 3 matrices (4 mM histidine and 6 mM histidine respectively) appear to be practically identical. Estimation of the secondary structure contents is summarized in Table 3. The results suggest that ABX-IL8 in both formulations has around 69% β-sheet, which is typical of antibody structures analyzed by IR spectroscopy.

TABLE 3
Secondary Structure Contents of ABX-IL8 in Formulation
1 and 3 as Determined by FTIR Spectroscopy

| | | Samples | |
|---|---|---|---|
| Wavenumber (cm⁻¹) | | Formulation 1 | Formulation 3 |
| 1707-1685 | β-sheet | 21% | 22% |
| 1685-1657 | α-helix | 23% | 23% |
| 1657-1622 | β-sheet | 49% | 47% |

### EXAMPLE 10

### Preparation of Lyophilized Formulations for Comparison Between Histidine and Sucrose

ABX-IL8 antibodies were purified and buffer-exchanged into 8 different formulations (2⁷⁻⁴ fractional factorial design) using PD10 columns (Amersham Pharmacia Biotech, Uppsala, Sweden) as shown in Table 4. Each formulation of 4 mL was dispensed into 10-mL Type 1 glass vials with 13-mm lyophilization stoppers. Lyophilization was carried out in a LyoStar freeze-dryer (FTS Systems, Stone Ridge, N.Y.). The samples were reconstituted with 1 mL water for injection (WFI). The lyophilized cakes were stored at 40° C. for 2 months. The percentage of aggregates in the samples were analyzed with SEC-HPLC and turbidity measurements.

TABLE 4
Eight Formulations Based on the 2⁷⁻⁴ Fractional Factorial Design Matrix for
Comparison between Histidine and Sucrose.

| | Factors^{a} | | | | | | | |
|---|---|---|---|---|---|---|---|---|
| | Antibody | Sucrose | Glycine | Maltose | Arginine | Mannitol | Histidine | Response^{b} |
| # | (mg/mL) | (mM) | (mM) | (mM) | (mM) | (mM) | (mM) | % Agg. |
| 1 | 50 | 15 | 10 | 10 | 10 | 10 | 10 | 4.65 |
| 2 | 50 | 15 | 15 | 10 | 15 | 15 | 15 | 3.03 |
| 3 | 50 | 10 | 10 | 15 | 10 | 15 | 15 | 3.63 |
| 4 | 50 | 10 | 15 | 15 | 15 | 10 | 10 | 4.11 |
| 5 | 30 | 15 | 10 | 15 | 15 | 10 | 15 | 2.14 |
| 6 | 30 | 15 | 15 | 15 | 10 | 15 | 10 | 2.06 |
| 7 | 30 | 10 | 10 | 10 | 15 | 15 | 10 | 2.38 |
| 8 | 30 | 10 | 15 | 10 | 10 | 10 | 15 | 2.62 |

^{a}Excipient concentrations were the concentrations in bulk drug solutions. All formulations contain 0.025% polysorbate 20.
^{b}The lyophilized cakes were stored at 40° C. for 2 months and analyzed with SEC-HPLC.

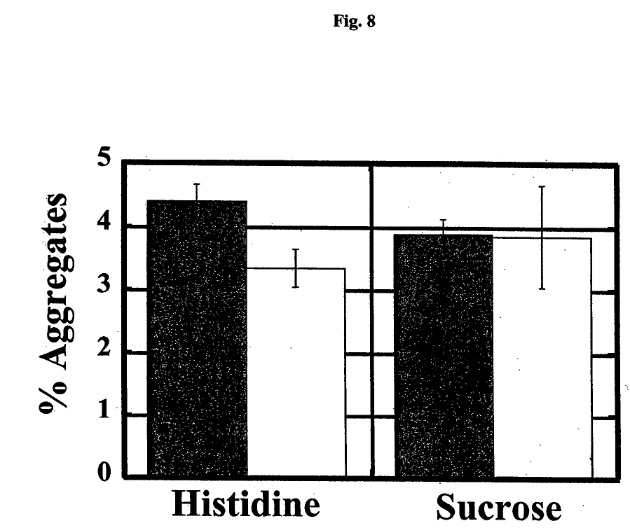

FIG. 8 is a bar graph that compares the effect of histidine and sucrose on the formation of soluble aggregates determined by SEC-HPLC. ABX-IL8 was lyophilized from bulk solutions containing 50 mg/mL ABX-IL8 in 10 mM (solid bars) or 15 mM (hollow bars) concentrations of histidine or sucrose. Referring to FIG. 8, the solid bars represent 10 mM histidine or sucrose formulations and the hollow bars indicate 15 mM histidine or sucrose formulations. The histidine hollow bar represents the average percentage of aggregation of Formulations 2 and 3 from Table 4. The histidine solid bar represents the average of Formulations 1 and 4 from Table 4. At 15 mM histidine and 50 mg/mL antibody, the molar ratio of histidine to ABX-IL8 is 45:1. Analysis of the data indicated that at the molar ratio of 45:1 (excipient:antibody), histidine conferred an equivalent protective effect on the antibody as sucrose.

### EXAMPLE 11

### Preparation and Storage of Liquid Formulations

The purified antibody was formulated into 11 different formulations (Table 5) using a TFF system with Biomax 30 membrane (Millipore, Bedford, Mass.). Each formulation of 0.8 mL was dispensed into 3-mL Type 1 glass vials with 13-mm serum stoppers. These samples, containing different levels of histidine, were studied in Example 12, 13, 14.

TABLE 5
Formulations for Solution Stability Studies

| Formulation | Composition | Note |
|---|---|---|
| A^{a} | 40 mM histidine, 40 mM arginine, | 50 mM less of |
| | | sucrose than B |
| | 150 mM sucrose | 25 mM more of |
| | | histidine and 25 mM |
| | | more of arginine |
| | | (total 50 mM) |
| B | 15 mM histidine, 15 mM arginine, | 50 mM more of |
| | | sucrose than A |
| | 200 mM sucrose | 25 mM less of |
| | | histidine and 25 mM |
| | | less of arginine |
| | | (total 50 mM) |
| C^{b} | 5 mM histidine, pH 6.0 | Excipient:antibody |
| | | ratio = 14 |
| D | 15 mM histidine, pH 6.0 | Excipient:antibody |
| | | ratio = 41 |
| E | 40 mM histidine, pH 6.0 | Excipient:antibody |
| | | ratio = 109 |
| F | 60 mM histidine, pH 6.0 | Excipient:antibody |
| | | ratio = 164 |
| G | 101 mM histidine, pH 6.0 | Excipient:antibody |
| | | ratio = 275 |
| H | 138 mM histidine, pH 6.0 | Excipient:antibody |
| | | ratio = 376 |
| I | 15 mM citrate, pH 6.0 | Excipient:antibody |
| | | ratio = 14 |
| J | 15 mM succinate, pH 6.0 | Excipient:antibody |
| | | ratio = 14 |

^{a}Formulation A and B contain 100 mg/mL ABX-IL8.
^{b}Formulation C to J contain 55 mg/mL ABX-IL8

### EXAMPLE 12

### Comparison Between Sucrose and Histidine in Liquid Formulations

A comparison between the stabilizing effects of histidine and sucrose on liquid antibody formulations was conducted. ABX-IL8 was formulated into two formulations, Formulation A and B (Table 5). Both Formulation A and B consisted of histidine, arginine, sucrose and polysorbate 20 with ABX-IL8 concentration at 100 mg/mL. The difference between the two formulations was that Formulation A contained 50 mM more histidine/arginine (25 mM histidine/25 mM arginine) than Formulation B, which contained 50 mM more sucrose than Formulation A.

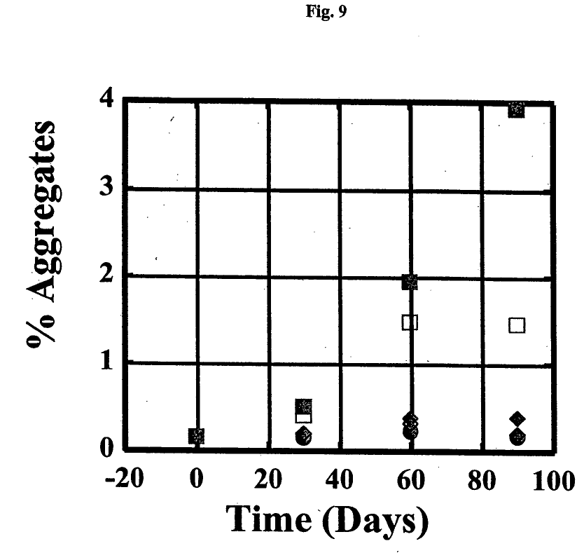

Both formulations were stored at 2-8, 25 and 40° C. for 3 months. The stability profile was checked every month. The resulting data is provided in a point graph in FIG. 9. FIG. 9 shows that ABX-IL8 in Formulation B (solid symbols) had higher level of soluble aggregates than that in Formulation A (hollow symbols) when samples were stored at 25° C. and 40° C., with samples at 40° C. having more pronounced and higher levels of aggregates. Circles represent samples stored at 2-8° C., diamonds represent samples stored at 25° C. and squares represent samples stored at 40° C. The results suggest that histidine combined with arginine confer a better protective effect than sucrose, at the levels tested.

### EXAMPLE 13

### Measuring Aggregation in Relation to Histidine Concentration

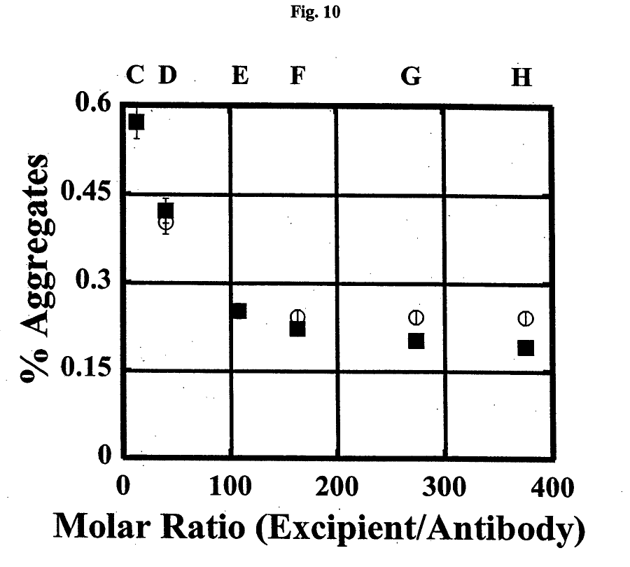

The percentage of aggregation in six formulations with various levels of histidine was measured using SEC-HPLC. From Table 5, Formulations C, D, E, F, G, and H with 5 mM, 15 mM, 40 mM, 60 mM, 101 mM and 138 mM of histidine, respectively, were measured for percentage of aggregation. Samples were frozen at −70° C. and thawed at room temperature for three cycles and assayed with SEC-HPLC. The results were compared to formulations having varying sucrose concentrations and lacking histidine. The results, are provided in FIG. 10. FIG. 10 is a point graph that shows the correlation between aggregation percentage and molar ratio of excipient to antibody. The six squares represent (from left to right) Formulations C, D, E, F, G, and H from Table 5. The circles represent formulations with sucrose instead of histidine. The results demonstrate that histidine is as effective as sucrose in stabilizing antibodies under freezing stress, indicating its ability to provide cyroprotection.

### EXAMPLE 14

### Histidine Stability Profile

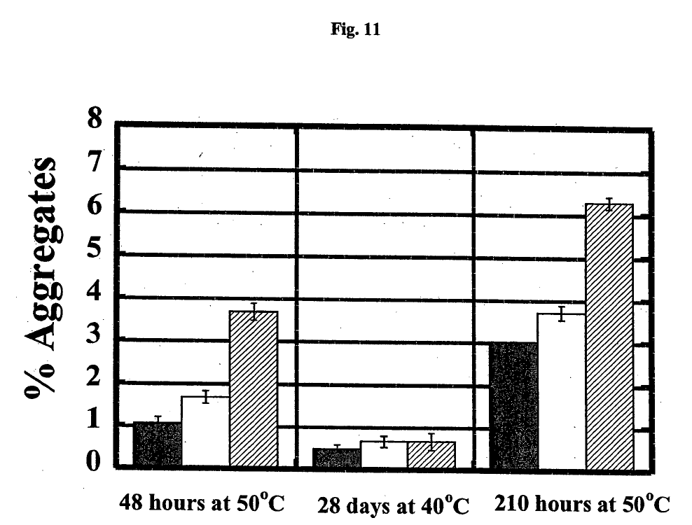

An antibody/histidine liquid formulation (Formulation D from Table 5) was compared to two other antibody containing liquid formulations (Formulations I and J from Table 5) containing citrate and succinate respectively. The percentage of aggregation for each formulation was measure after 28 days at 40° C., 48 hours at 50° C., and 210 hours at 50° C. The results are presented in FIG. 11. FIG. 11 is a bar graph that illustrates histidine's effectiveness in preventing aggregation in liquid antibody containing formulations. With reference to Table 5, the solid bar represents Formulation D (15 mM histidine), the hollow bar represents Formulation I (15 mM citrate) and the striped bar represents Formulation J (15 mM succinate). The results demonstrate that formulations containing histidine had lower antibody aggregates than those formulations containing citrate or succinate at the same pH.

### EXAMPLE 15

### Liquid Formulation Stability

The stability profile of a liquid formulation containing 100 mg/mL antibody, 40 mM histidine, 40 mM arginine, 150 mM sucrose, 0.04% polysorbate 20 was measured. Specifically, the percent of monomers in each formulation was measured and recorded over various periods of time and temperatures. The results are provided below in Table 6.

TABLE 6
Stability profile of the liquid formulation of ABX-IL8

| Time point (month) | Storage Temp. (° C.) | pH | % Monomer |
|---|---|---|---|
| 6 | 2-8 | 6.0 | 99.8 |
| 6 | 25 | 6.0 | 99.5 |
| 3 | 40 | 6.0 | 98.4 |

The results show that the antibody formulation is fairly stable even at relatively high temperatures. After storage at 25° C., which is more higher than the recommended storage temperature of 2-8° C., for 6 months, the purity of the antibody remained at 99.5% monomer. Based on the Arrhenius plot extrapolation, the predicted shelf life of the dosage form, t₉₅ (purity of 95% will remain) will be greater than 24 months at 2-8° C.

Furthermore, the effect of small but deliberate variations in the formulation parameters such as excipient concentrations on the quality of the antibody were also tested. The results show that the liquid formulation is robust. Variations of histidine concentrations from 15 mM to 60 mM, arginine concentrations from 15 mM to 60 mM, sucrose concentrations from 100 mM to 200 mM, and polysorbate 20 concentrations from 0.01 to 0.1% did not affect the overall quality of the product.

### EXAMPLE 16

### Solution Viscosity Studies

Solution viscosity is a very important property for an antibody formulation. Due to the fact that proteins or antibodies tend to reversibly associate, the formulations containing higher concentrations of the antibody will become viscous, which makes it difficult to scale-up and manufacture the dosage form. Accordingly, any means that can effectively reduce the solution viscosity of a formulation containing high concentration of a protein would be desirable.

The solution viscosity of ABX-IL8 liquid formulations containing different levels of histidine was tested.

Purified ABX-IL8 antibody was concentrated using a TFF system with Biomax 30 membrane to 150 mg/mL in 5 mM histidine, pH 6. Stock solutions of histidine alone (0.5 M) or histidine/arginine (0.5 M/0.5M) were aliquoted and concentrated under vacuum in a Speed-Vac (Savant Instruments, Farmingdale, N.Y.). Concentrated salt was spiked into antibody solution to different concentrations for viscosity measurement. The volume change at the highest spiking concentration was less than 5 percent. Therefore, the antibody concentration was maintained upon spiking of the salts, which was confirmed by measurement of the concentrations using A₂₈₀. Viscosity measurements was carried out at room temperature using Cannon-Fenske capillary viscometer (Brinkmann, Westbury, N.Y.).

Solution samples were dispensed into an appropriate sized capillary using a 10 mL syringe. The capillary loaded with sample solution was secured in a vertical holder. The solution was allowed to flow freely down past two marks. The amount of time it took a given sample to flow from the upper mark to the lower mark was recorded in seconds as the efflux time. The kinematic viscosity of the solution was calculated by multiplying the efflux time by the constants.

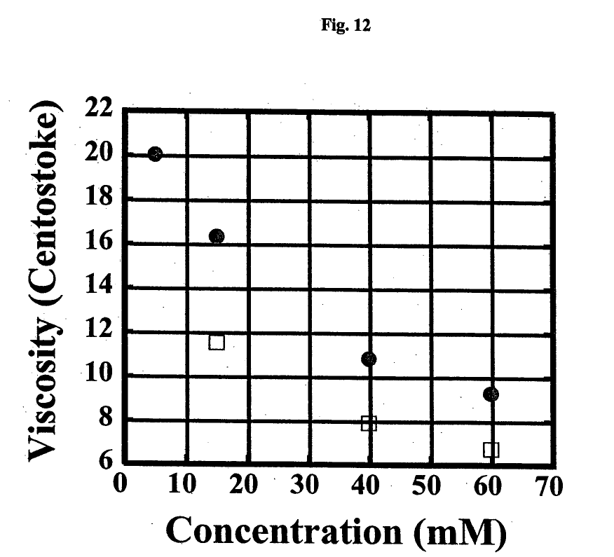

FIG. 12 is a point graph that shows the effect of histidine (solid circle) or histidine/arginine (hollow square) on the solution viscosity of ABX-IL8 antibody. The formulations contained different concentrations of excipients (histidine or histidine/arginine) from 5 mM to 60 mM. The resulting data demonstrates that increasing histidine levels in the formulations led to decreases of viscosity in a concentration-dependent manner. Addition of arginine in the histidine-containing formulations further reduced the solution viscosity.

### EXAMPLE 17

### Titration Study and Results

Histidine was spiked into a formulation matrix consisting of 17.5 mM glycine, 0.25% mannitol, 18.8 mM glutamic acid and 0.025% polysorbate 20, to final histidine concentrations of 5 mM, 15 mM and 40 mM, all at pH 6. Samples were freeze-dried using two different cycles: Cycle II and Cycle III.

The Cycle II freeze-drying protocol was performed as follows. The shelf was precooled to −45° C. Primary drying occurred at −20° C. with a ramping rate of 0.5° C./min from −45° C. to −20° C. and then held for 75 hours at a chamber pressure of 70 mTorr for 75 hours. Secondary drying followed at 20° C. with a ramping rate of 0.5° C./min from −20° C. to 20° C. and held for 44 hours at a chamber pressure of 50 mTorr. The total cycle time was approximately 120 hours. The dried cakes were stored at 40° C. for two weeks.

The Cycle III freeze-drying protocol was performed as follows. Samples were frozen at a rate of 0.35° C./min to −45° C. Primary drying occurred at 20° C. with a ramping rate of 0.16° C./min from −45° C. to 20° C. and then held for 25 hours at a chamber pressure of 200 mTorr. Secondary drying followed at 30° C. with a ramping rate of 0.5° C./min and held for 10 hours at a chamber pressure of 50 mTorr. The total cycle time was approximately 50 hours. The dried cakes were stored at 40° C. for two weeks.

The percentage of aggregates was then measured. The results (Table 7) indicate that the antibody was more stable in the formulation matrix containing higher histidine concentration, upon lyophilization and storage, regardless of the freeze-drying cycle. The pH of the formulation did not account for the stabilization of the antibody because it was fixed at 6 in the study.

TABLE 7
Titration Study and Results

| Formulation | | % Aggregates | % Aggregates |
|---|---|---|---|
| No. | Components^{a} | Cycle II | Cycle III |
| 1 | 17.5 mM Glycine, 0.25% | 3.24 ± 0.18 | 3.23 ± 0.16 |
| | mannitol, 18.8 mM | | |
| | glutamic acid, 0.025% | | |
| | polysorbate 20, 5 mM | | |
| | histidine, pH 6 | | |
| 2 | 17.5 mM Glycine, 0.25% | 2.21 ± 0.04 | 1.63 |
| | mannitol, 18.8 mM | | |
| | glutamic acid, 0.025% | | |
| | polysorbate 20, 15 mM | | |
| | histidine, pH 6 | | |
| 3 | 17.5 mM Glycine, 0.25% | 1.89 | 1.58 ± 0.01 |
| | mannitol, 18.8 mM | | |
| | glutamic acid, 0.025% | | |
| | polysorbate 20, 40 mM | | |
| | histidine, pH 6 | | |

^{a}The concentrations of excipients represent those in bulk solution prior to lyophilization

All publications and patent documents cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A solid formulation comprising at least one antibody, and histidine in a sufficient amount to stabilize said at least one antibody in said solid formulation.

2. The solid formulation of claim 1, further comprising an excipient.

3. The solid formulation of claim 2, wherein said at least one other excipient is selected from the group consisting of: mannitol, Polysorbate 20, Polysorbate 80, succinate, citrate, Tris, phosphate, sucrose, trehalose, amino acids, polyols, PEG, BSA, sucrose, lactose, maltose, and sorbital.

4. The solid formulation of claim 2, wherein said at least one other excipient is arginine.

5. The solid formulation of claim 1, wherein said at least one antibody is a mammalian antibody.

6. The solid formulation of claim 1, wherein said at least one antibody is a human antibody.

7. The solid formulation of claim 1, wherein said at least one antibody is a human monoclonal IgG2 antibody.

8. The formulation of claim 1, wherein the sufficient amount of histidine is between 6 and 40 mM.

9. The formulation of claim 1, wherein the sufficient amount of histidine is about 15 mM of histidine.

10. A method of preparing an antibody in a solid formulation comprising:
mixing at least one antibody with a stabilizing amount of histidine to form a mixture; and
treating said mixture to generate a solid formulation of said antibody and said histidine.

11. The method of claim 10, wherein treating said mixture comprises lyophilizing said mixture.

12. The method of claim 10, wherein said solid formulation is a lyophilized cake.

13. The method of claim 11, wherein lyophilizing said mixture comprises:
freezing said mixture at a rate of about −0.35° C. per minute until said mixture reaches a temperature of about −45° C.; and
sufficiently drying said mixture.

14. The method of claim 13, wherein drying comprises a primary and a secondary drying.

15. The method of claim 12, further comprising reconstituting said lyophilized cake with a reconstituting agent.

16. The method of claim 15, wherein said reconstituting agent comprises water for injection (WFI).

17. The method of claim 10, further comprising adding at least one other excipient to said mixture.

18. The method of claim 17, wherein said at least one other excipient is selected from the group consisting of: mannitol, Polysorbate 20, Polysorbate 80, succinate, citrate, Tris, phosphate, trehalose, amino acids, polyols, PEG, BSA, sucrose, lactose, maltose, and sorbital.

19. The method of claim 15, wherein said at least one other excipient is arginine.

20. The method of claim 10, wherein the stabilizing amount of histidine is between 6-40 mM.

21. The method of claim 10, wherein the stabilizing amount of histidine is about 15 mM.

22. The method of claim 11, wherein lyophilizing said mixture occurs in less than 100 hours.

23. The method of claim 11, wherein lyophilizing said mixture occurs in less than 50 hours.

24. The method of claim 11, wherein lyophilizing said mixture occurs in about 45 hours.

25. A kit for preparing a solid formulation of a stabilized antibody comprising;
a first container, comprising at least one antibody in solution, and
a second container comprising a sufficient amount of histidine in solution to stabilize said antibody when said antibody is dried into a solid formulation.

26. The kit of claim 25, wherein said antibody is a mammalian antibody.

27. The kit of claim 25, wherein said antibody is a human antibody.

28. The kit of claim 25, wherein said antibody is a human monoclonal IgG₂ antibody.

29. The kit of claim 25, wherein the sufficient amount of histidine is between 6-40 mM.

30. The kit of claim 25, wherein the sufficient amount of histidine is about 15 mM.

31. A liquid formulation comprising at least one antibody, and histidine in a sufficient amount to stabilize said at least one antibody in said liquid formulation.

32. The liquid formulation of claim 31, further comprising an excipient.

33. The liquid formulation of claim 32, wherein said at least one other excipient is selected from the group consisting of: mannitol, Polysorbate 20, Polysorbate 80, succinate, citrate, Tris, phosphate, sucrose, trehalose, amino acids, polyols, PEG, BSA, sucrose, lactose, maltose, and sorbital.

34. The liquid formulation of claim 32, wherein said at least one other excipient is arginine.

35. The liquid formulation of claim 31, wherein said at least one antibody is a mammalian antibody.

36. The liquid formulation of claim 31, wherein said at least one antibody is a human antibody.

37. The liquid formulation of claim 31, wherein said at least one antibody is a human monoclonal IgG₂ antibody.

38. The liquid formulation of claim 31, wherein the sufficient amount of histidine is between 6 and 40 mM.

39. The formulation of claim 31, wherein the sufficient amount of histidine is about 15 mM of histidine.
